(19) United States
(12) Patent Application Publication
(10) Pub. No.: US 2020/0298178 A1
(43) Pub. Date: Sep. 24, 2020

(54) Portable Moisture Absorbing Athletic Bag

(71) Applicant: Jorge Cuesta, Katy, TX (US)

(72) Inventor: Jorge Cuesta, Katy, TX (US)

(21) Appl. No.: 16/359,833

(22) Filed: Mar. 20, 2019

(51) Int. Cl. B01D 53/28; A45C 3/00; A45C 13/02; A61L 9/012

(52) U.S. Cl. CPC B01D 53/28; A45C 3/001; A45C 13/02; B01D 53/261; A45C 2003/007; A45C 2013/026; A61L 9/012

(57) ABSTRACT

A tote bag with an internal drying feature accepts sweaty exercise clothing or shoes for temporary storage and dries such items after exercise and during working hours until the contents can be brought to a laundry facility. The drying agent can be regenerated with a heat source or other ways. Additional materials can be combined with a drying agent for odor control such as baking soda. The drying agent can feature an indicator that provides feedback on the remaining useful life such as by a color change. The drying agent can be sized to fit in internal pouches in the bag that can optionally be secured to retain the drying agent in place. An array of drying agent elements supported with a backing material can be used to expedite drying agent replacement.

### FIELD OF THE INVENTION

The field of the invention is portable tote bags or containers with a moisture absorbing feature and more particularly bags that can be used to carry sweaty athletic clothing or shoes while controlling odor within the bag as the items inside are dried.

### BACKGROUND OF THE INVENTION

Drying agents such as silica gel are known and used in a variety of applications such as in packaging for electronic equipment to keep moisture under control. Some applications to clothing have been used to control moisture in the form of sweat while the clothing is worn such as during outdoor activities. Some examples of this are US 20070174949 and US 20160255897. Some designs feature a container for the moisture absorbing material that removes moisture from a surrounding space such as a closet as in US 20080314772. Another application is a bedding mat for sweat absorption from the body as in U.S. Pat. No. 5,092,008. A wound care application is described in US 2009009488. Some containers are built to dehumidify the surrounding air as opposed to the internal contents as in US 20120061261.

The present invention addresses a late developing need for those people who exercise during normal working hours and have a need to store their sweaty exercise clothes in their office or workspace until the end of the work day when the soiled clothing can be brought home and laundered. The clothing and shoes used in daily exercise during the working day become more pungent as the day wears on. The storage container of the present invention contains odors from such clothing or shoes and dries such articles during the working day. In this manner when it is time to launder such items the odor is less offensive as the drying agent has been at work during the work day and after use to remove moisture from such articles. The moisture removing agent can cooperate with auxiliary drying agents and can further contain an indicator reflecting remaining ability to further remove moisture. The drying agent can be thermally rejuvenated or reactivated for continuing applications. The container or tote can either have the drying agent permanently built-in (sewed to the inside walls) or be modular such that the drying agent can be exchanged and replaced for continued service. These and other aspects of the present invention will be more readily apparent to those skilled in the art from a review of the detailed description of the preferred embodiment and the associated drawings while recognizing that the full scope of the invention is to be determined from the literal and equivalent scope of the appended claims.

### SUMMARY OF THE INVENTION

A tote bag with an internal drying feature accepts sweaty exercise clothing or shoes for temporary storage and dries such items after exercise and during working hours until the contents can be brought to a laundry facility. The drying agent can be regenerated with a heat source or other ways. Additional materials can be combined with a drying agent for odor control such as baking soda. The drying agent can feature an indicator that provides feedback on the remaining useful life such as by a color change. The drying agent can be sized to fit in internal pouches in the bag that can optionally be secured to retain the drying agent in place. An array of drying agent elements supported with a backing material can be used to expedite drying agent replacement. The drying agent can be thermally rejuvenated or reactivated for continuing applications. The container or tote can either have the drying agent permanently built-in (sewed to the inside walls) or be modular such that the drying agent can be exchanged and replaced for continued service. Optionally, the backing material can be modular segments that are selectively connected to form the container for exercise clothes or shoes.

### BRIEF DESCRIPTION OF THE DRAWINGS

FIG. 1 is an outside view of the bag showing a closure for the bag opening;

FIG. 2 is an inside view of opposed internal walls of the bag in a laid flat presentation;

FIG. 3 is a section view through an internal wall shown in FIG. 2;

FIGS. 4 and 5 schematically illustrate the use of a color indicator on the drying agent indicating a fully activated condition in FIG. 4 and a spent condition in FIG. 5;

FIGS. 6 and 7 schematically illustrate the drying agent combined with an odor control material or a fragrant material, respectively;

FIG. 8 illustrates sweaty articles inserted into the opening of the bag at 11 AM; and

FIG. 9 is the view of FIG. 8 at a later time in the afternoon schematically showing moisture removed from the articles at, for example, 4:30 PM.

### DETAILED DESCRIPTION OF THE PREFERRED EMBODIMENT

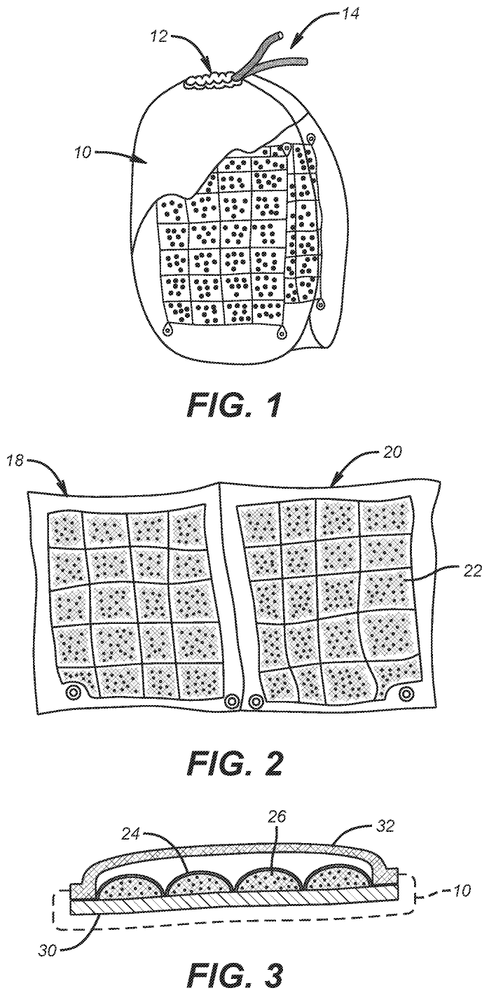

FIG. 1 shows a bag 10 with an opening 12 and a closure 14 for the opening 12 that can be a draw string, a zipper or interlocking fibers, for example. A brand name or other forms of advertising can go on one or opposed outer walls such as 16. FIG. 2 shows a possible inside detail illustrating modular segments 18 and 20. Segments 18 and 20 are shown in section in FIG. 3. A square grid pattern 22 is shown that is composed of a porous material that passes moisture 24 and inside each compartment that defines the pattern is a drying material 26 that is preferably silica gel but can be other materials that grab moisture from the air or soiled clothing or shoes 28. Although a square grid pattern is shown with a single piece of drying material 26 in a discrete compartment, other arrangements are contemplated. While the arrangement is preferably optimized for outer surface area of the drying agent 26 or any porous cover over the drying agent 26 to minimize crumbling or dusting other arrays are contemplated keeping in mind the space needed inside the bag 10 for articles 28 and the need to target the empty weight of the bag for the users convenience. The porous pockets or spaces 24 can be formed on an impermeable backing material 30 that can be fabric, to save weight. Thus the backing material 30 stays connected to the porous pockets or spaces 24 with the drying agent 26 inside. A porous liner material 32 can be mounted to the backing material 30 for tandem removal from the bag 10 or the porous liner material 32 can be secured internally to the bag 10 so that it stays in the bag when the assembly of items 24, 26 and 30 are removed from the bag 10 for regeneration such as with a heat source such as a microwave oven, preferably on low power or a normal oven with the setting at under 300 degrees Fahrenheit or in a clothes dryer. If the assembly of items 24, 26 and 30 is not removable the bag 10 should be turned inside out before regenerating the drying agent 26. The regenerating temperature used can vary with the selected materials, with those skilled in the art appreciating that the time to regenerate the drying agent 26 varies inversely with the temperature of the heat source. Alternatively, the FIG. 3 assemblies, of which there are two disposed in an opposed manner, can be left in the bag 10 and the entire bag 10 with the insert opposed assemblies such as in FIG. 3 can be regenerated as a unit without removing the FIG. 3 opposed assemblies from the bag 10. In this option the backing assemblies 30 can actually form the outer walls of the bag 10 as the entirety of the bag 10 is regenerated as a unit. FIG. 3 shows that the outer wall 10 can be attached to the liner material 32 to define a pocket or space in between so that the assembly of 24, 26 and 30 can be removed as a unit for regeneration. Alternatively, items 10 and 30 can be one and the same so that the assembly of 24, 26 and 30 remain an integral part of the bag for regenerating the drying agent 26 as a unit.

The drying agent 26 can be presented in a variety of shapes such as hemispherical, disc or other geometric shape. For ease of manufacturing all the shapes can be identical but that is optional. The end surfaces or at least one end surface can be planar or arcuate but shapes that enhance the outer surface area are preferred as this has a direct effect on its ability to adsorb moisture. This can be accomplished with irregular end surfaces such as arrays of protrusions spaced apart from interspersed valleys, akin to a waffle shape as schematically illustrated in FIGS. 10 and 11, for example.

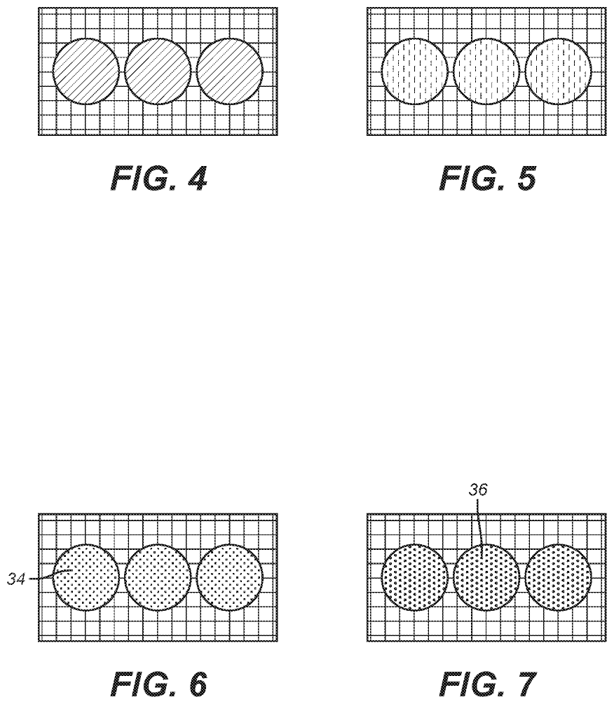

FIGS. 4 and 5 schematically illustrate the use of cobalt chloride which is blue when dry and light purple when moisture saturated. Methyl violet can also be used. Methyl violet is commonly used on pH test strips. This material is orange when dry and green when fully saturated. Either of these indicators suggest the timetable for regeneration of the drying agent 26 as the colors vary between the stated colors for the dry to the saturated condition. In addition or in the alternative, FIGS. 6 and 7 illustrate the use of a material to control odor such as baking soda 34 that can be either adhered to the drying agent 26 or manually added periodically on an as needed basis. By the same token an air freshening material 36 can be adhered to the drying agent 26 or periodically manually added as needed.

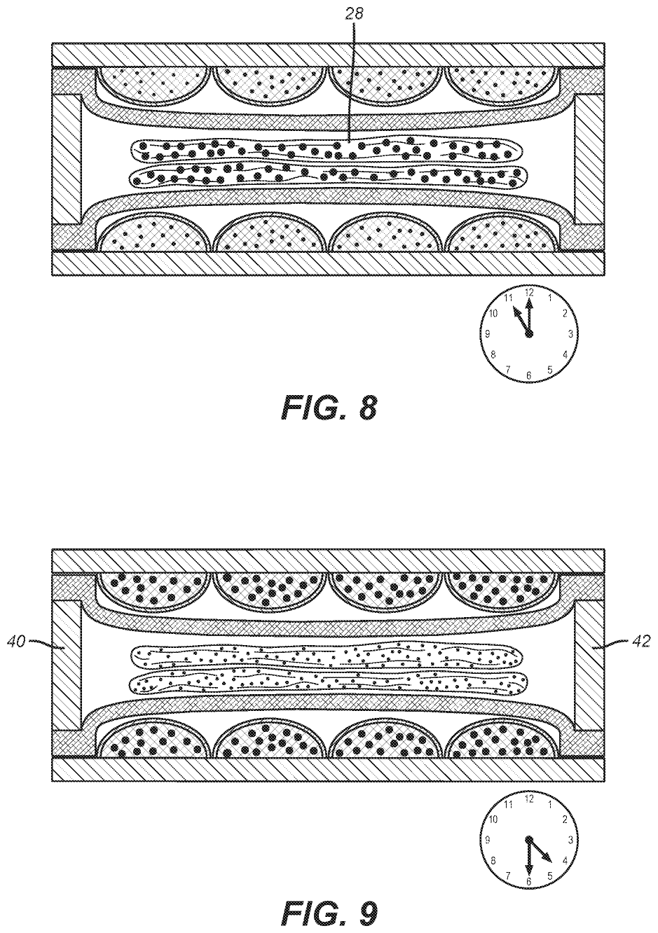

FIGS. 8 and 9 shows the array of FIG. 3 in opposed facing modules so that the articles of clothing or shoes 28 can be put into the bag 10 in between and closed at closure 12. The moisture is removed from the clothing or shoes 28 and into the drying agent 26 while odors are retained in the bag 10 and mold and mildew is controlled with periodic regeneration of the drying agent 26 in the manner described above.

Pockets or spaces 24 can be selectively opened to replace a drying agent 26 mounted inside. In that manner individual elements, regardless of their shape, can be replaced if they reach the point where regeneration becomes ineffective. Alternatively, an entire array such as 24, 26 and 30 can be replaced as an assembly. The opposed arrays as depicted in FIGS. 3, 8 and 9, can be spaced apart by sidewalls such as 40 and 42 which give the bag 10 width to hold soiled clothing or sweaty shoes. The walls 40 and 42 can also have drying agent 26 disposed in the manner previously described for the opposed long walls such as 16.

Those who face a long interval between athletic activity and access to laundry facilities such as people that exercise before or during the work day will find the bag and described method for storage of soiled clothing or shoes particularly helpful. The stored items are dried while stored in a container that retains or masks odors. The drying process controls mold or mildew formation and facilitates the laundering process that takes place after working hours. The benefits ultimately include the long term preservation of the athletic clothing. Color changing materials provide an indication that the drying agent is approaching a spent condition. Regeneration of the drying agent can occur with removal of panels of the drying agent from the bags for application of heat from a conventional dryer, a microwave or standard oven or some other heat source. If the regeneration temperature is controlled it is also possible to leave the drying agent (as in the version with permanent array) in the bag and apply heat to the empty bag without disassembly of internal components. The outer walls that form the bag are moisture impervious such that the drying effect takes place within the bag and is directed at the contents of the bag without taking moisture from outside the bag. The bag can have rigid exterior walls to hold a shape to facilitate loading the bag. Material that controls odor or adds fragrance can be used in tandem with the drying agent.

The drying agent can be silica gel, zeolite or activated alumina, although silica gel is preferred because it absorbs a great deal of moisture by adsorption and regenerates at fairly low temperatures of about 180 degrees Fahrenheit. It can absorb as much as 40% moisture by volume.

The wall material for the bag may have a small transparent window to alert the user that it has contents that need attention, so they do not get overlooked.

The above description is illustrative of the preferred embodiment and many modifications may be made by those skilled in the art without departing from the invention whose scope is to be determined from the literal and equivalent scope of the claims below:

I claim:

1. A storage container for soiled clothing or footwear items until a laundry facility becomes subsequently available, comprising:
an impermeable wall structure defining a selectively closable opening through which said clothing or footwear items are inserted or removed;
a drying agent disposed within said impermeable wall structure to remove moisture from said clothing or footwear items while said clothing or footwear items are stored therein.

2. The storage container of claim 1, wherein:
said impermeable wall structure comprises at least one permeable liner defining at least one space with said impermeable wall structure, said drying agent disposed in said space.

3. The storage container of claim 2, wherein:
said drying agent is removably mounted in said space.

4. The storage container of claim 2, wherein:
said drying agent is disposed in an array of compartments in said space.

5. The storage container of claim 4, wherein:
said compartments are selectively closable.

6. The storage container of claim 4, wherein:
said array is removable from said space with said drying agent as an assembly.

7. The storage container of claim 4, wherein:
said array is removable with said permeable liner through said opening.

8. The storage container of claim 1, wherein:
said drying agent further comprises a color changing material to indicate the remaining ability of the drying agent to remove moisture from the soiled clothing or footwear items.

9. The storage container of claim 1, wherein:
said drying agent comprises an odor control agent or a fragrance material.

10. The storage container of claim 1, wherein:
said drying agent comprises multiple spaced apart units supported by said impermeable wall structure in an array said units configured to maximize external surface area for enhancing moisture absorption.

11. The storage container of claim 10, wherein:
said units of said drying agent comprise at least one geometric shape of silica gel.

12. The storage container of claim 11, wherein:
said at least one shape comprises a plurality of protrusions and depressions to enhance surface area for moisture adsorption from said soiled clothing and footwear items.

13. The storage container of claim 11, wherein:
said units of said drying agent removably mounted to said impermeable wall structure for regeneration apart from said impermeable wall structure.

14. The storage container of claim 11, wherein:
said units of said drying agent non-removably mounted to said impermeable wall structure for regeneration together with said impermeable wall structure.

15. A method of temporary storage of soiled clothing or footwear between use and laundering, comprising:
providing a container further comprising an impermeable wall structure defining a selectively closable opening through which said clothing or footwear items are inserted or removed;
inserting the clothing or footwear into said container; providing a drying agent disposed within said impermeable wall structure to remove moisture from said clothing or footwear items while said clothing or footwear items are stored therein;
removing the clothing or footwear for laundering.

16. The method of claim 15, wherein:
providing at least one permeable liner defining at least one space with said impermeable wall structure, said drying agent disposed in said space.

17. The method of claim 16, wherein:
removably mounting said drying agent in said space.

18. The method of claim 16, wherein:
disposing said drying agent in an array of compartments in said space.

19. The method of claim 15, wherein:
said drying agent further comprises a color changing material to indicate the remaining ability of the drying agent to remove moisture from the soiled clothing or footwear items.

20. The method of claim 15, wherein:
said drying agent comprises an odor control agent or a fragrance material.
